(19) United States
(12) Patent Application Publication
(10) Pub. No.: US 2024/0395368 A1
(43) Pub. Date: Nov. 28, 2024

(54) METHODS, DEVICES, AND NON-TRANSITORY COMPUTER STORAGE MEDIUM OF MATCHING CLINICAL TRIALS

(71) Applicant: TAIPEI MEDICAL UNIVERSITY, TAIPEI CITY (TW)

(72) Inventors: CHENG-YU CHEN, TAIPEI CITY (TW); SHIH-HSIN HSIAO, TAIPEI CITY (TW); YUNG-CHUN CHANG, TAIPEI CITY (TW)

(21) Appl. No.: 18/321,005

(22) Filed: May 22, 2023

(51) Int. Cl. G16H 10/20; G16H 50/70

(52) U.S. Cl. CPC G16H 10/20; G16H 50/70

(57) ABSTRACT

Disclosed are methods, devices and the non-transitory computer storage media of matching clinical trials. The present disclosure provides a method of matching clinical trials. The method comprises: obtaining a first data set from a pathology report; obtaining a second data set of a clinical trial; determining whether the first data set and the second data set are matched with respect to a first set of fields; determining a relevance value between the first data set and the second data set with respect to a second set of fields when the first data set and the second data set are matched with respect to the first set of fields; and determining the clinical trial as recommended when the relevance value exceeds a threshold.

### FIELD OF THE INVENTION

The present disclosure relates to a method of matching clinical trials, a device of matching clinical trials, and a related non-transitory computer storage media. In particular, the present disclosure relates to methods of matching clinical trials for a patient based on the pathology report thereof, and to related devices and non-transitory computer storage medium.

### BACKGROUND

A pathology report of a patient includes a large amount of information, especially for cancer patients, and such pathology report includes a substantial amount of miscellaneous and tedious information. The surgeon and the physician in charge may spend much time to understand a patient's situation and to find a clinical trial, which may be suitable for the patient, but computers may be helpful in reducing the amount of time wasted and thus may increase overall efficiency.

### SUMMARY OF THE INVENTION

The subject disclosure can analyze a pathology report of a patient and find a suitable clinical trial for the patient. A pathology report may contain the diagnosis determined by examining cells and tissues under a microscope. The pathology report may be for a lung cancer patient. Important messages can be summarized from a miscellaneous and tedious pathology report. Such messages may include categories of features: basic description in pathology, tumor features, histological description, immunohistochemistry (IHC) information, a genetic testing result, and a pathological TNM (tumor, node and metastasis) stage. The present disclosure can further summarize multiple pathology reports of one patient. The present disclosure can further provide a function of collecting data of a large amount of clinical trials, and compare features obtained from the pathology report with the clinical trials to determine the suitable clinical trials for the patient, which can be a reference for the surgeon and the physician.

An embodiment of the present disclosure provides a method of matching clinical trials. The method comprises: obtaining a first data set from a pathology report; obtaining a second data set of a clinical trial; determining whether the first data set and the second data set are matched with respect to a first set of fields; determining a relevance value between the first data set and the second data set with respect to a second set of fields when the first data set and the second data set are matched with respect to the first set of fields; and determining the clinical trial as recommended when the relevance value exceeds a threshold.

Another embodiment of the present disclosure provides a device of matching clinical trials. The device comprises a processor and a memory coupled with the processor. The processor executes computer-readable instructions stored in the memory to perform operations, and the operations comprise: obtaining a first data set from a pathology report; obtaining a second data set of a clinical trial; determining a relevance value between the first data set and the second data set with respect to a first set of fields; and determining the clinical trial is recommended when the relevance value exceeds a threshold.

A further embodiment of the present disclosure provides a non-transitory computer storage medium. The non-transitory computer storage medium has program instructions stored thereon. Upon execution of the program instructions by a processor, the program instructions cause performance of a set of operations. The operations comprises: obtaining a first data set from a pathology report; obtaining a second data set of a clinical trial; determining whether the first data set and the second data set are matched with respect to a first set of fields; determining a relevance value between the first data set and the second data set with respect to a second set of fields when the first data set and the second data set are matched with respect to the first set of fields; and determining the clinical trial is recommended when the relevance value exceeds a threshold.

### BRIEF DESCRIPTION OF THE DRAWINGS

In order to describe the manner in which advantages and features of the present disclosure can be obtained, a description of the present disclosure is rendered by reference to specific embodiments thereof, which are illustrated in the appended drawings. These drawings depict only example embodiments of the present disclosure and are not therefore to be considered limiting of its scope.

FIG. 1 illustrates a schematic diagram of a system of matching clinical trials according to some embodiments of the present disclosure.

FIG. 2 illustrates a flow diagram of the secondary condition matching included in the clinical trial matching system in FIG. 1 according to some embodiments of the present disclosure.

FIG. 3 illustrates a flow diagram of a method of matching clinical trials according to some embodiments of the present disclosure.

FIG. 4A illustrates a flow diagram of a method of pre-training a model for extracting features from a pathology report according to some embodiments of the present disclosure.

FIG. 4B illustrates a flow diagram of a method of extracting features from a pathology report according to some embodiments of the present disclosure.

FIG. 5 illustrates a flow diagram of a method 50 of collecting clinical trials according to some embodiments of the present disclosure.

FIG. 6 illustrates a schematic diagram of a representation of a clinical trial matching system according to some embodiments of the present disclosure.

FIG. 7 illustrates a schematic diagram showing a computer system in accordance with some embodiments of the present disclosure.

### DETAILED DESCRIPTION

The following disclosure provides many different embodiments, or examples, for implementing different features of the provided subject matter. Specific examples of operations, components, and arrangements are described below to simplify the present disclosure. These are, of course, merely examples and are not intended to be limiting. For example, a first operation performed before or after a second operation in the description may include embodiments in which the first and second operations are performed together, and may also include embodiments in which additional operations may be performed between the first and second operations. For example, the formation of a first feature over, on or in a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Time relative terms, such as “prior to,” “before,” “posterior to,” “after” and the like, may be used herein for ease of description to describe one operation or feature's relationship to another operation(s) or feature(s) as illustrated in the figures. The time relative terms are intended to encompass different sequences of the operations depicted in the figures. Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Relative terms for connections, such as “connect,” “connected,” “connection,” “couple,” “coupled,” “in communication,” and the like, may be used herein for ease of description to describe an operational connection, coupling, or linking one between two elements or features. The relative terms for connections are intended to encompass different connections, coupling, or linking of the devices or components. The devices or components may be directly or indirectly connected, coupled, or linked to one another through, for example, another set of components. The devices or components may be wired and/or wirelessly connected, coupled, or linked with each other.

As used herein, the singular terms “a,” “an,” and “the” may include plural referents unless the context clearly indicates otherwise. For example, reference to a device may include multiple devices unless the context clearly indicates otherwise. The terms “comprising” and “including” may indicate the existences of the described features, integers, steps, operations, elements, and/or components, but may not exclude the existences of combinations of one or more of the features, integers, steps, operations, elements, and/or components. The term “and/or” may include any or all combinations of one or more listed items.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

The nature and use of the embodiments are discussed in detail as follows. It should be appreciated, however, that the present disclosure provides many applicable inventive concepts that can be embodied in a wide variety of specific contexts. The specific embodiments discussed are merely illustrative of specific ways to embody and use the disclosure, without limiting the scope thereof.

To match a pathology report of a patient (e.g., a pathology report of lung cancer) with clinical trials, the present disclosure provides a method of extracting the pathological features from a pathology report. In some embodiments of the present disclosure, a pathology report may include pathological features among categories. Exemplary pathological features are listed in Table 1. The categories may include: the basic description, the finding (of tumor(s)), the histology (information of tumor(s)), the IHC information, the genetic testing (result), and the TNM stage. In further embodiments, the data representation of report(s) of a patient may be represented by the pathological features shown in Table 1.

TABLE 1

| Category | Pathological Features |
|---|---|
| Basic | organ, Bx-site, sampling method, diagnosis |
| Description | |
| Finding | greatest dimension, tumor size, closest margin, |
| | lymphovascular invasion, VPI, tumor focality |
| Histology | histology type, histology grade |
| IHC | CK7, TTF-1, Napsin A, CK20, P40, CDX2, P63, P16, |
| | cytokeratin(AE1/AE3), Vimentin, PAX-8, CD56, |
| | chromogranin-A, synaptophysin, GATA3, P53, S100, |
| | Ki67, EBER, Her2 |
| Genetic | EGFR, ALK, ROS1, KRAS, BRAF, RET, NTRK |
| testing | (Neurotrophic Tyrosine Receptor Kinase), MET, ERBB2, |
| | PIK3CA, NRAS, MEK1, PDL1 |
| TNM stage | version, pT, pN, pM, pStage, N info |

The present disclosure provides a clinical trials matching system, which can analyze a pathology report including pathological features and the demographic data (i.e., the personal information of the patient). The clinical trials matching system can determine the similarity and relevance between said pathology report and the clinical trials, and then find out the recommended clinical trials for the patient. The recommended clinical trials can be a reference for the surgeon and the physician. Therefore, the surgeon and the physician can provide more options of treatment for the patient based on the recommended clinical trials. Using the clinical trials matching system, the surgeon and the physician can find the suitable clinical trial quickly and accurately, without lots of time searching clinical trials manually.

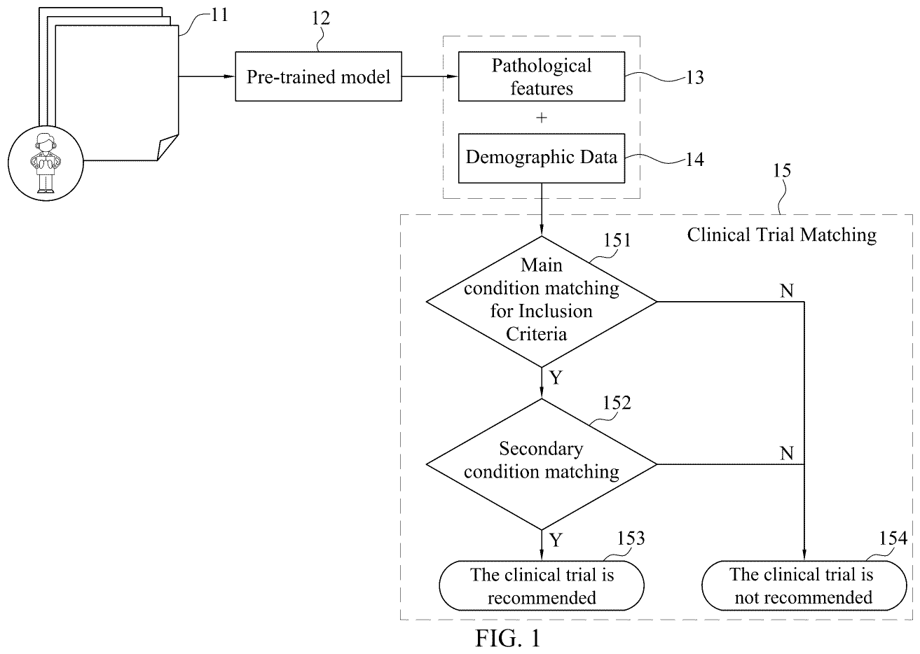

FIG. 1 illustrates a schematic diagram of a system of matching clinical trials according to some embodiments of the present disclosure.

Referring to FIG. 1, a pathology report 11 of a patient can be provided. The pathology report 11 can be input to a pre-trained model 12 to extract one or more pathological features 13. The pathological features 13 can be provided to a clinical trial matching system 15.

The pre-trained model 12 can perform a classification task and/or a sequence tagging task to extract or obtain the pathological features 13. The pathological features 13 may include the information related to EGFR, ALK, ROS1, KRAS, BRAF, RET, NTRK, MET, P53, and Her2 and the information related to operations (e.g., surgical operations), histology, a tumor size, a stage (e.g., pathologic staging), and PDL1. Through the classification task, the information related to EGFR, ALK, ROS1, KRAS, BRAF, RET, NTRK, MET, P53, Her2, and etc. may be extracted or obtained. Regarding the sequence tagging task, the information related to operations, histology, tumor size, stage, PDL1, and etc. may be extracted or obtained.

In some embodiments, a demographic data 14 of the patient can be provided to the clinical trial matching system 15. The demographic data 14 may include data or information related to age, gender, smoking, nodal metastases, distant metastases, CNS metastases, bone metastases, wild type, anti-angiogenesis, platinum, EGFR TKIs, ALK inhibitors, PD-1/PD-L1 inhibitors, CTLA-4 inhibitor, radiotherapy, cisplatin/carboplatin, chemotherapy, systemic therapy, disease status, ECOG PS, and etc. The demographic data 14 may be extracted or obtained through the classification task or the sequence tagging task of the pre-trained model 12. The demographic data 14 may be obtained through accessing the relevant database when the demographic data 14 is stored in a format (e.g., computer-processible data) which can be directly utilized by the clinical trial matching system 15.

The clinical trial matching system 15 can analyze the pathological features 13 and the demographic data 14 of the patient, and find out one or more suitable clinical trials for the patient. In some embodiments, the clinical trial matching system 15 can coupled to a clinical trials database (not shown), such that the clinical trial matching system 15 can compare the pathological features 13 and the demographic data 14 with the clinical trials. Accordingly, the recommended clinical trial could be found for the patient.

Referring to FIG. 1, the clinical trial matching system 15 includes operations 151, 152, 153, and 154. The pathological features 13 and the demographic data 14 of the patient can be input into the clinical trial matching system 15 and be compared with each clinical trial. In operation 151, one or more main conditions (or fields) in the pathological features 13 and the demographic data 14 are determined whether they match the inclusion criteria of a clinical trial. In some embodiments, the one or more main conditions in the pathological features 13 and the demographic data 14 are exactly matched with the inclusion criteria of the clinical trial. Said main conditions (or fields) may include at least one of: estimated glomerular filtration rate (EFGR), surgical operation, histology, pathologic staging, age, gender, or smoking. In some embodiment, at operation 151, when the values or information of a set of conditions of the patient are exactly matched with those of the set of conditions recited in the inclusion criteria, the process will proceed to operation 152. The set of conditions may include the EGFR condition, the surgical operation condition, the histology condition, the pathologic staging condition, the age condition, the gender condition, and the smoking condition.

When the one or more main conditions are exactly matched, the process will proceed to operation 152. On the other hand, when the one or more main conditions are not exactly matched, the process will proceed to operation 154.

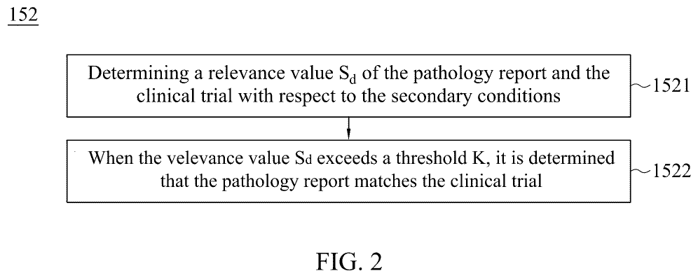

In operation 152, one or more secondary condition (or fields) in the pathological features 13 and the demographic data 14 are determined whether they match the clinical trial. In some embodiments, the one or more secondary conditions in the pathological features 13 and the demographic data 14 are partially matched. Different from the main conditions, the secondary conditions may not need to be exactly matched with the clinical trial. In some embodiments, the secondary conditions matching is determined by the relevance between the secondary conditions in the pathology report and in the clinical trial. In some embodiments, when the relevance value is greater than a threshold value, the one or more secondary conditions may be determined to match the clinical trial. The details of the determination of the relevance will be discussed in FIG. 2.

When the one or more secondary conditions are matched, the process will proceed to operation 153. On the other hand, when the one or more secondary conditions are not matched, the process will proceed to operation 154.

In operation 153, when the pathological features 13 and the demographic data 14 of the patient match the clinical trial (i.e., passing the operations 151 and 152), the clinical trial will be recommended for the patient. Then, the clinical trial matching system 15 may perform a further process to determine whether another clinical trial matches with the pathological features 13 and the demographic data 14 of the same patient.

In operation 154, when the pathological features 13 and the demographic data 14 of the patient do not match the clinical trial (i.e., not passing the operation 151 or 152), the clinical trial will be not recommended for the patient. Then, the clinical trial matching system 15 may perform a further process to determine whether another clinical trial matches with the pathological features 13 and the demographic data 14 of the same patient.

Utilizing the clinical trial matching system 15, the doctor can easily find the related in-process clinical trials for the patients. The clinical trial matching system 15 can filter clinical trials and thus can help surgeon and the physician to recommend the suitable clinical trials for the patient, so that the patient can have more treatment options.

FIG. 2 illustrates a flow diagram of the secondary condition matching included in the clinical trial matching system 15 in FIG. 1 according to some embodiments of the present disclosure. Referring to FIG. 2, the operation 152 of the secondary condition matching can include two steps 1521 and 1522.

In some embodiments, the secondary condition matching in the operation 152 is determined by the relevance between the pathology report and the clinical trial with respect to the secondary conditions. In step 1521, a relevance value S_{d} of the pathology report and the clinical trial is determined with respect to the secondary conditions (fields). In some embodiments, the pathology report can include the pathological features 13 and the demographic data 14 of the patient. In some embodiments, the relevance value S_{d} can be determined based on BM25 algorithm, which is a ranking function used to estimate the relevance of documents to a given search query.

In some embodiments, the relevance value S_{d} of the pathology report and a clinical trial d can be calculated by Eq. 1:

$${S}_{d} = {\sum }_{q ∈ Q} {W}_{q} \cdot ( {\sum }_{t ∈ q} [ log \frac{N}{{df}_{t}} ] \cdot \frac{( {k}_{1} + 1 ) {tf}_{t d}}{{k}_{1} ( 1 - b ) + b \times ( {L}_{d} / ( {L}_{a v g} ) ) + {tf}_{t d}} \cdot \frac{( {k}_{3} + 1 ) {tf}_{t d}}{{k}_{3} + {tf}_{t d}} ) , \qquad [ Eq . 1 ]$$

in which
Q represents all queries (e.g., the secondary conditions);
q represents an individual query (e.g., a secondary condition);
W_{q} represents an individual weight assigned to the individual query q;

$$log \frac{N}{{df}_{t}}$$

represents a respective inverse document frequency (IDF) for the respective keyword t;
dfₜ represents the number of the clinical trials including the respective keyword t;
N represents the number of the clinical trials in the clinical trials database;
tf_{td} represents the occurrence number of the keyword t in the clinical trial d;
L_{d} represents the length of the clinical trial d;
L_{avg} represents the average length of all clinical trials in the clinical trial database;
k₁ is a constant for normalizing the range of the frequency for the keyword in a document (for example, k₁ can be in a range of 1.2 to 2.0, preferably, 1.2 or 1.5)
k₃ is a constant for correcting the range of the frequency for the keywords in the query (for example, k₃ can be in a range of 1.2 to 2.0, preferably, 1.2 or 1.5); and
b is a constant (for example, b can be 0.75 or 0.5).

In some embodiments, an individual relevance value can be obtained from one query (e.g., one field of the second set of fields or one condition of the secondary conditions), and the relevance value S_{d} is a sum of the individual relevance values. The Eq. 1 can include the individual weight W_{q}, the respective inverse document frequency (IDF), a similarity between the respective keyword t and the individual query q, and a weight of the respective keyword t. The respective IDF can be expressed as

$$log \frac{N}{{df}_{t}} .$$

The similarity between the respective keyword t and the individual query q can be expressed as

$$\frac{( {k}_{1} + 1 ) {tf}_{t d}}{{k}_{1} ( 1 - b ) + b \times ( {L}_{d} / ( {L}_{a v g} ) ) + {tf}_{t d}} .$$

In some embodiments, the similarity equation includes (k₁+1) due to the use of Laplace smoothing.

The weight of the respective keyword t can be expressed as

$$\frac{( {k}_{3} + 1 ) {tf}_{t d}}{{k}_{3} + {tf}_{t d}} .$$

In some embodiments, the weight of the respective keyword t includes (k₃+1) due to the use of Laplace smoothing.

In some embodiments, the individual relevance value can be associated with the individual weight W_{q}, which is assigned to the individual query by the clinician. In particular, the individual relevance value can be proportional to the individual weight W_{q}. The clinician can determine the importance or relevance of each query (or condition) and then assign a proper weight to such query (or condition).

In some embodiments, the IDF is a numerical statistic that is intended to reflect how important a word/term is to a document in a collection or corpus. The IDF is a weight that indicates how commonly the word/term is used. The more frequent its usage across documents in a collection or corpus, the lower its IDF score. The lower the IDF score, the less important the word/term becomes. For example, the term “the” appears in almost all English texts and thus would have a very low IDF score since the term carries very little “topic” information.

In some embodiments, the individual relevance value can be associated with a respective IDF for a respective keyword. For example, the individual relevance value can be proportional to the respective IDF. Accordingly, the respective keyword appears less across clinical trials in the clinical trial database, the greater the respective IDF thereof would be, and thus the individual relevance value would be greater.

Utilizing the Eq. 1, the relevance value S_{d} of the pathology report and the clinical trial can be determined with respect to the secondary conditions (fields).

In step 1522, when the relevance value S_{d} exceeds a threshold K, it is determined that the pathology report (or the corresponding pathological features 13 and demographic data 14) matches the clinical trial. Back to FIG. 1, when the pathological features 13 and the demographic data 14 matches the clinical trial at operation 152, the clinical trial is then recommended for the corresponding patient at operation 153. The relevance value S_{d} can be compare with the threshold K, such that the clinical trial can be determined whether it matches the pathology report. When the relevance value S_{d} of the clinical trial exceeds the threshold K, the clinical trial is recommended for the patient (i.e., going to the operation 153 in FIG. 1) On the other hand, when the relevance value S_{d} between the pathology report and the clinical trial is less than the threshold K, it is determined that the pathology report (or the corresponding pathological features 13 and demographic data 14) does not match the clinical trial. Back to FIG. 1, when the pathological features 13 and the demographic data 14 does not match the clinical trial at operation 152, the clinical trial is not recommended for the corresponding patient at operation 154.

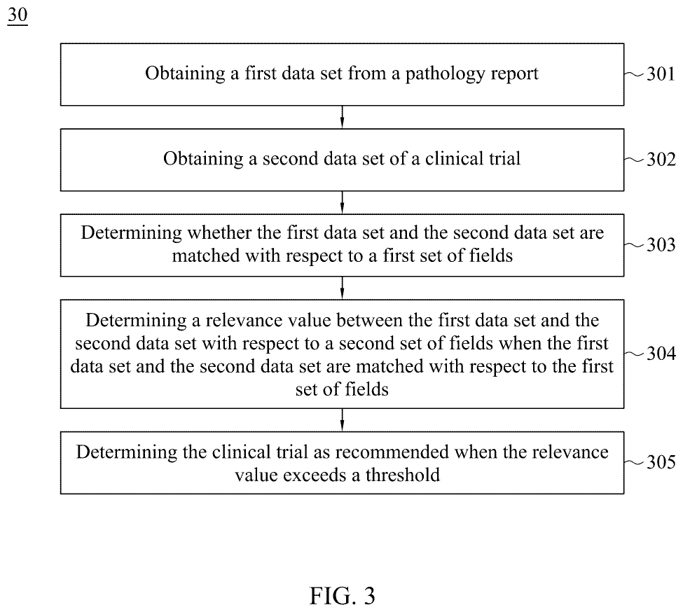

FIG. 3 illustrates a flow diagram of a method 30 of matching clinical trials according to some embodiments of the present disclosure.

In operation 301, a first data set can be obtained from a pathology report. In some embodiments, the first data set can include the pathological features 13 and the demographic data 14 of the patient as discussed in FIG. 1. For example, the first data set (such as the demographic data 14) can be obtained from the pathology report. In another embodiment, the first data set (such as the pathological features 13) can be obtained from the pathology report through the pre-trained model 12.

In operation 302, a second data set of a clinical trial can be obtained. In some embodiments, the second data set of the clinical trial can be obtained from the clinical database.

In operation 303, whether the first data set and the second data set are matched with respect to a first set of fields can be determined. In some embodiments, the operation 303 may correspond to the operation 151 in FIG. 1. The first set of fields can include one or more of: estimated glomerular filtration rate (EFGR), surgical operation, histology, pathologic staging, age, gender, or smoking.

In operation 304, a relevance value between the first data set and the second data set with respect to a second set of fields can be determined when the first data set and the second data set are matched with respect to the first set of fields. In some embodiments, the operation 304 may correspond to the operation 152 in FIG. 1. The second set of fields can include one or more of: ALK, ROS1, KRAS, BRAF, RET, NTRK, MET, P53, Her2, tumor size, tumor maximum diameter, programmed death-ligand 1 (PD-L1), nodal metastases, Distant metastases, CNS metastases, bone metastases, wild type, Anti-angiogenesis, Platinum, EGFR TKIs, ALK inhibitors, PD-1/PD-L1 inhibitors, CTLA-4 inhibitor, Radiotherapy, cisplatin/carboplatin, Chemotherapy, systemic therapy, Disease status, or Eastern Cooperative Oncology Group Performance Status (ECOG PS). In operation 305, the clinical trial can be determined as recommended when the relevance value exceeds a threshold. When the relevance value of the clinical trial exceeds the threshold, it indicates that the clinical trial is relevant to the patient, and thus the clinical trial can be recommended for the patient.

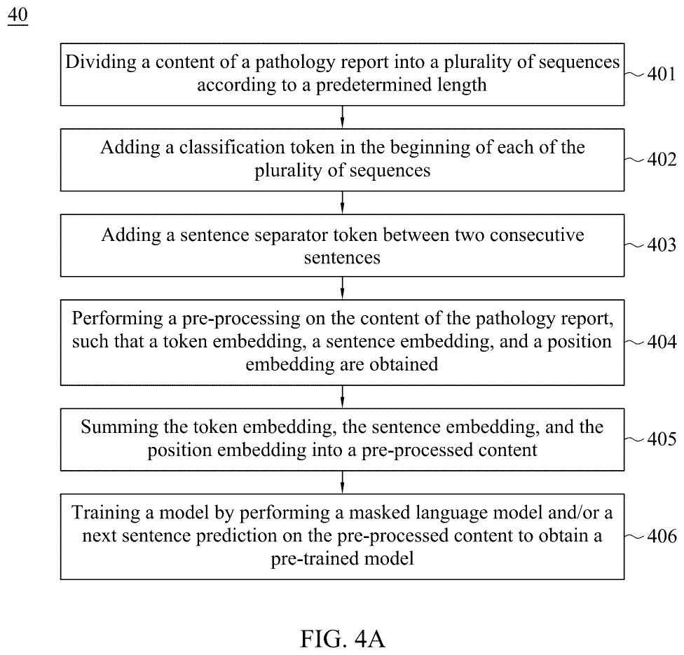

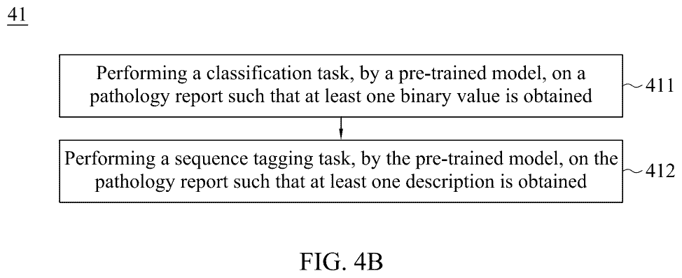

FIG. 4A illustrates a flow diagram of a method 40 of pre-training a model for extracting features from a pathology report according to some embodiments of the present disclosure. In some embodiments, the model can be pre-trained by input unlabeled wording contents, such as pathology reports of several patients.

In operation 401, a content of a pathology report can be divided into a plurality of sequences according to a predetermined length. In some embodiments, each of the sequences can include a plurality of sentences.

In operation 402, a classification token can be added in the beginning of each of the plurality of sequences. In some embodiments, the sequences of the pathology report can be one or more paragraphs, which can be identified by clinicians. The classification token can representing the vector of the whole sequence.

In operation 403, a sentence separator token can be added between two consecutive sentences. In some embodiments, the sentence separator token can be used to identify different sentences. In some embodiments, each of the two consecutive sentences can include one or more sentences identified by clinicians.

In operation 404, a pre-processing can be performed on the content of the pathology report, such that a token embedding, a sentence embedding, and a position embedding are obtained. In some embodiments, the token embedding can be the value representation of the content. The sentence embedding can be the value representation of the sentence. The position embedding can be the position representation of the content.

In operation 405, the token embedding, the sentence embedding, and the position embedding can be summed into a pre-processed content. In some embodiments, the token embedding, the sentence embedding, and the position embedding can be summed, the summed content is duplicated in three copies and then a multi-head self-attention algorithm can be performed on the three copies, and the pre-processed content containing the representation vector of each wording can be obtained.

In operation 406, a model can be trained by performing a masked language model and/or a next sentence prediction on the pre-processed content to obtain a pre-trained model.

In some embodiments, the masked language model can trivially predict the target term in a multi-layered context. The masked language model is that some portions of the input term (such as some wordings of the specific term) can be simply masked randomly, and then those masked terms can be predicted. In some embodiments, the input terms can be transformed into tokens for analysis.

In some embodiments, to train a model understanding sentence relationship, the next sentence prediction can be used to train the model. In some embodiments, the next sentence prediction a task that trivially generated from a corpus/database. Specifically, when choosing the sentences A and B for each pertaining example, 50% of the time B is the actual next sentence that follows A, and 50% of the time it is a random sentence from the corpus. After trained with large input data, the accuracy of the next sentence prediction of the pre-trained model can increase.

In some embodiments, the pre-trained model can be a Bidirectional Encoder Representations from Transformers (BERT) model. The pre-trained model can be pre-trained by inputting clinical pathology reports of one or more hospitals.

FIG. 4B illustrates a flow diagram of a method 41 of extracting features from a pathology report according to some embodiments of the present disclosure. In some embodiments, the method 41 can be performed by the pre-trained model trained according to the method 40 in FIG. 4A.

In operation 411, a classification task can be performed, by the pre-trained model, on a pathology report such that at least one state value is obtained. In some embodiments, the classification task is to determine whether the pathology report includes the specific field or not. Therefore, the answer/result of the classification task would be yes or no (1 or 0). That is, the result of the classification task is a state value.

In some embodiments, the at least one state value in the pathology report can includes the state values for the fields (or conditions) including: EGFR, ALK, ROS1, KRAS, BRAF, RET, NTRK, MET, P53, or Her2. In some embodiments, the at least state value can be included in the first data set discussed in FIG. 3.

For example, the state value of the EGFR field may include 2⁴30 1 possible values, e.g., mutation states of exons 18, 19, 20, and 21 and an unknown state. For the fields of ALK, ROS1, KRAS, BRAF, RET, NTRK, MET, P53, and Her2, the state value may be positive, negative, or unknown.

In operation 412, a sequence tagging task can be performed, by the pre-trained model, on the pathology report such that at least one description is obtained. In some embodiments, the sequence tagging task can determine the specific term for different categories. Therefore, the answer/result of the sequence tagging task would be a description.

In some embodiments, the at least one description in the pathology report includes the description for the fields (or conditions) including: operation (or surgical operation), histology, tumor size, stage (or pathologic staging), or PDL1. In some embodiments, the at least description can be included in the first data set discussed in FIG. 3.

For example, the description of the operation field can be “VATS (Video-Assisted Thoracic Surgery) lobectomy.” The description of the histology field can be “poorly differentiated non-small cell carcinoma.” The description of the tumor size field can be “0.6×0.4×0.3 cm,” and the description of the maximum tumor diameter can be “0.6 cm.” he description of the stage field can be “pStage IVA.”

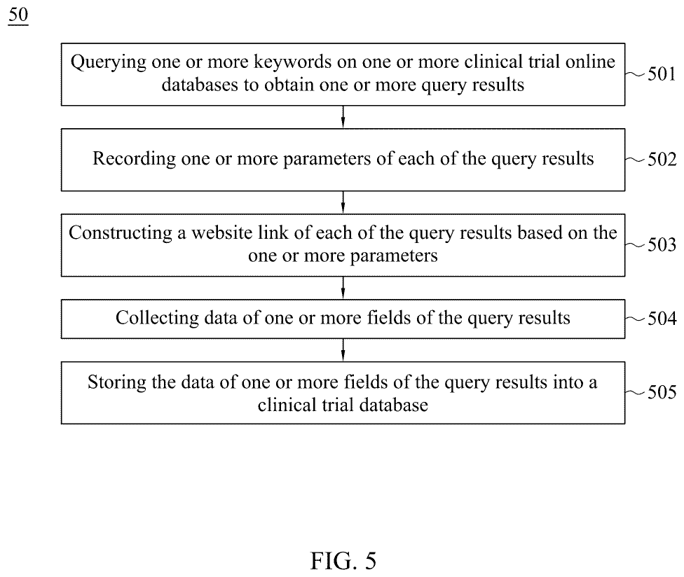

FIG. 5 illustrates a flow diagram of a method 50 of collecting clinical trials according to some embodiments of the present disclosure. The device performing method 50 can be for update the clinical trial database, which may be coupled with the clinical trials matching system in FIG. 1.

In operation 501, one or more keywords can be queried on one or more clinical trial online databases to obtain one or more query results. The one or more clinical trial online databases can be a government public clinical trial database (such as, clinicaltrials.gov and www1.cde.org.tw/ct_taiwan). In some embodiments, the keywords can be disease/diagnosis and/or stage. In some embodiments, the disease/diagnosis can include NSCLC (Non-Small Cell Lung Cancer), non-small cell, lung adenocarcinoma, non-squamous, squamous cell carcinoma, non-squamous non-small cell lung cancer, squamous cell lung cancer, large cell lung cancer . . . , and etc. In some embodiments, the stage can include Advances, Stage IIIB, Stage IIIC, Stage IV, Metastatic . . . , and etc. For example, the keywords can be NSCLC Advanced, NSCLC Stage IIIB . . . , and etc.

In operation 502, one or more parameters of each of the query results can be recorded.

In operation 503, a website link of each of the query results can be constructed based on the one or more parameters.

In operation 504, data of one or more fields of the query results can be collected. In some embodiments, the fields can be the interested columns in the query results. For example, the fields can include the keyword, the ID of clinical trials/programs, the clinical trial/project title, applicant, sponsor, the estimated start date of the clinical trial, the actual start date of the clinical trial, the estimated end date of the clinical trial, the actual end date of the clinical trial, the inclusion criteria and the exclusion criteria of the clinical trial, the trial hospital, the trial locations (such as, state or country), the estimated trial number in Taiwan, the estimated trial number in the world, the last updated date of the clinical trial, and the website link of the clinical trial (i.e., url).

In operation 505, the data of one or more fields of the query results can be stored into a clinical trial database. In some embodiments, the clinical trial database can be coupled with the clinical trials matching system, such that the system can compare the pathological features and the demographic data with the clinical trials. Accordingly, the recommended clinical trial could be found for the patient.

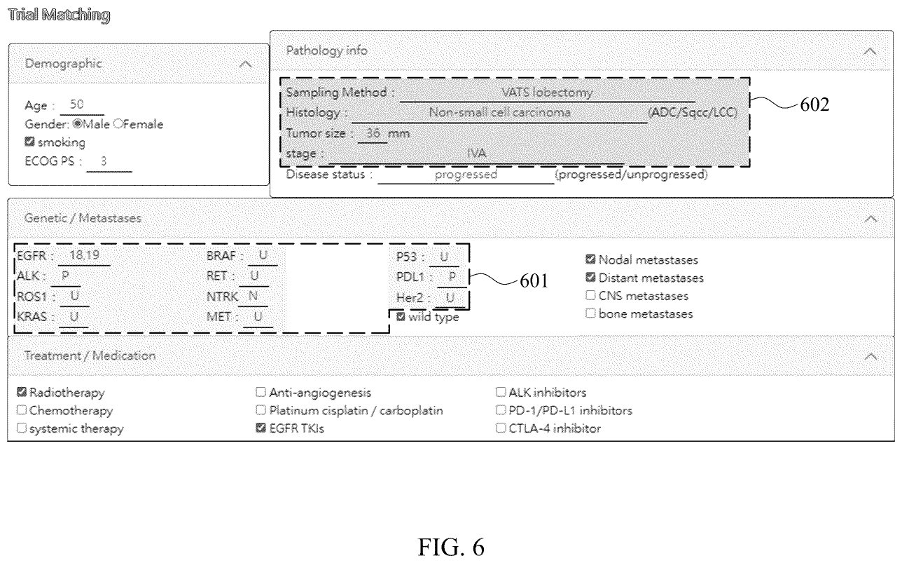

FIG. 6 illustrates a schematic diagram of a representation of a clinical trial matching system according to some embodiments of the present disclosure. Referring to FIG. 6, the clinical trial matching system can include a demographic block, genetic/metastases block, treatment/medication block, and pathology information block.

The data in the demographic block, such as age, gender, smoking, and ECOG PS, can be obtained from the demographic data 14 in FIG. 1. For example, the age can be 50. The gender can be male. The patient can have smoking habit. The ECOG PS can have a score of 3, which may be ranged from 0 to 5.

In some embodiments, the data 601 in the genetic/metastases block can be obtained from the pathology report through the pre-trained model. That is, the data 601 can be obtained from the pathological features 13 in FIG. 1. On the other hand, the data other than the data 601 in the genetic/metastases block can be obtained from the demographic data 14 in FIG. 1. In some embodiments, each of the data in the genetic/metastases block can be a state value. For the EGFR field, it shows “unknown” or the number of exon having mutation; the state value “18, 19” indicates that exon 18 and 19 have mutation. For other fields in the data 601, the state value may be P (positive), N (negative), or U (unknown). For example, the nodal metastases can be yes (i.e., the nodal metastases has occurred).

In the genetic/metastases block, the state value of “wild type,” “nodal metastases,” “distant metastases,” “CNS metastases,” and “bone metastases” may be true or false.

The data in the treatment/medication block can be obtained from the demographic data 14 in FIG. 1. In some embodiments, each of the data in the treatment/medication block can be a state value, which may be true or false. For example, the radiotherapy can be yes (i.e., the radiotherapy has been conducted).

In some embodiments, the data 602 in the pathology information block can be obtained from the pathology report through the pre-trained model. That is, the data 602 can be obtained from the pathological features 13 in FIG. 1. On the other hand, the data other than the data 602 in the pathology information block can be obtained from the demographic data 14 in FIG. 1.

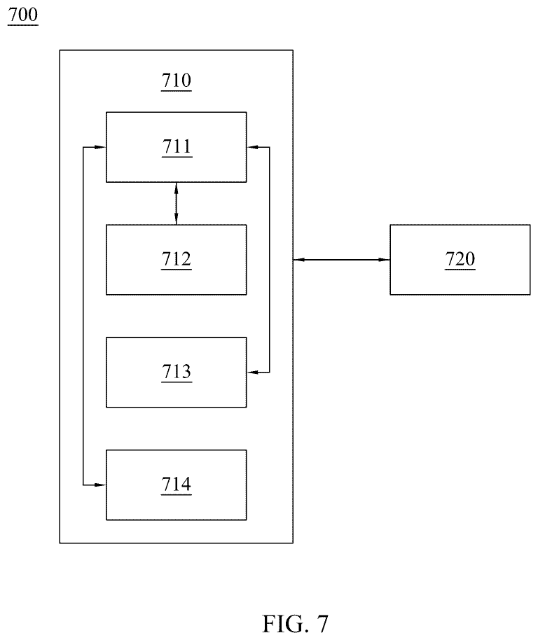

FIG. 7 illustrates a schematic diagram showing a computer system in accordance with some embodiments of the present disclosure.

Referring to FIG. 7, it shows an example of a computer system 700 capable of performing one or more operations of the methods of the present disclosure. The computer system 700 includes, in at least some embodiments of the present disclosure, a computing device 710 and a database 720. The computing device 710 may be a server computer, a client computer, a personal computer (PC), a tablet PC, a set-top box (STB), a personal digital assistant (PDA), a cellular telephone, or a smartphone. The computing device 710 comprises processor 711, input/output interface 712, communication interface 713, and memory 714. The database 720 may store pathology reports from which the pathological features 13 and the demographic data 14 would be extracted. The database 720 may store pathology reports to be analyzed or summarized. The input/output interface 712 is coupled with the processor 711. The input/output interface 712 allows the user to manipulate the computing device 710 in order to perform the operations or methods of the present disclosure (for example, the method disclosed in FIG. 3). The communication interface 713 is coupled with the processor 711. The communication interface 713 allows the computing device 710 to communicate with the database 720. The communication interface 713 may support one or more of the following protocols: Universal Serial Bus (USB), Ethernet, Bluetooth, IEEE 802.11, 3GPP Long-Term Evolution (LTE) (4G), and 3GPP New Radio (5G). A memory 714 may be a non-transitory computer readable storage medium. The memory 714 is coupled with the processor 711. The memory 714 has stored program instructions that can be executed by one or more processors (for example, the processor 711). Upon execution of the program instructions stored on the memory 714, the program instructions cause performance of the one or more operations of the methods disclosed in the present disclosure.

For example, the program instructions may cause the computing device 710 to perform a set of acts that at least include: obtaining a first data set from a pathology report; obtaining a second data set of a clinical trial; determining whether the first data set and the second data set are matched with respect to a first set of fields; determining a relevance value between the first data set and the second data set with respect to a second set of fields when the first data set and the second data set are matched with respect to the first set of fields; and determining the clinical trial as recommended when the relevance value exceeds a threshold.

The scope of the present disclosure is not intended to be limited to the particular embodiments of the process, machine, manufacture, and composition of matter, means, methods, steps, and operations described in the specification. As those skilled in the art will readily appreciate from the disclosure of the present disclosure, processes, machines, manufacture, composition of matter, means, methods, steps, or operations presently existing or later to be developed, that perform substantially the same function or achieve substantially the same result as the corresponding embodiments described herein may be utilized according to the present disclosure. Accordingly, the appended claims are intended to include within their scope, processes, machines, manufacture, and compositions of matter, means, methods, steps, or operations. In addition, each claim constitutes a separate embodiment, and the combination of various claims and embodiments are within the scope of the disclosure.

The methods, processes, or operations according to embodiments of the present disclosure can also be implemented on a programmed processor. However, the controllers, flowcharts, and modules may also be implemented on a general purpose or special purpose computer, a programmed microprocessor or microcontroller and peripheral integrated circuit elements, an integrated circuit, a hardware electronic or logic circuit such as a discrete element circuit, a programmable logic device, or the like. In general, any device on which resides a finite state machine capable of implementing the flowcharts shown in the figures may be used to implement the processor functions of present disclosure.

An alternative embodiment preferably implements the methods, processes, or operations according to embodiments of the present disclosure in a non-transitory, computer-readable storage medium storing computer programmable instructions. The instructions are preferably executed by computer-executable components preferably integrated with a network security system. The non-transitory, computer-readable storage medium may be stored on any suitable computer readable media such as RAMs, ROMs, flash memory, EEPROMs, optical storage devices (CD or DVD), hard drives, floppy drives, or any suitable device. The computer-executable component is preferably a processor, but the instructions may alternatively or additionally be executed by any suitable dedicated hardware device. For example, an embodiment of the present disclosure provides a non-transitory, computer-readable storage medium having computer programmable instructions stored therein.

While the present disclosure has been described with specific embodiments thereof, it is evident that many alternatives, modifications, and variations may be apparent to those skilled in the art. For example, various components of the embodiments may be interchanged, added, or substituted in the other embodiments. Also, all of the elements of each figure are not necessary for operation of the disclosed embodiments. For example, one of ordinary skill in the art of the disclosed embodiments would be enabled to make and use the teachings of the present disclosure by simply employing the elements of the independent claims. Accordingly, embodiments of the present disclosure as set forth herein are intended to be illustrative, not limiting. Various changes may be made without departing from the spirit and scope of the present disclosure.

Even though numerous characteristics and advantages of the present disclosure have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A method of matching clinical trials, comprising:
obtaining a first data set from a pathology report;
obtaining a second data set of a clinical trial;
determining whether the first data set and the second data set are matched with respect to a first set of fields;
determining a relevance value between the first data set and the second data set with respect to a second set of fields when the first data set and the second data set are matched with respect to the first set of fields; and
determining the clinical trial as recommended when the relevance value exceeds a threshold.

2. The method of claim 1, wherein the relevance value is a sum of an individual relevance value of each of the second set of fields.

3. The method of claim 2, wherein the individual relevance value is associated with an individual assigned weight (Wq).

4. The method of claim 2, wherein the individual relevance value is associated with a respective inverse document frequency (IDF) for a respective keyword.

5. The method of claim 1, wherein the first set of fields include one or more of: estimated glomerular filtration rate (EFGR), surgical operation, histology, pathologic staging, age, gender, or smoking.

6. The method of claim 1, wherein the second set of fields include one or more of: ALK, ROS1, KRAS, BRAF, RET, NTRK, MET, P53, Her2, tumor size, tumor maximum diameter, programmed death-ligand 1 (PD-L1), nodal metastases, distant metastases, CNS metastases, bone metastases, wild type, anti-angiogenesis, platinum, EGFR TKIs, ALK inhibitors, PD-1/PD-L1 inhibitors, CTLA-4 inhibitor, radiotherapy, cisplatin/carboplatin, chemotherapy, systemic therapy, disease status, or eastern cooperative oncology group performance status (ECOG PS).

7. The method of claim 1, wherein the obtaining the first data set comprises:
performing a classification task, by a pre-trained model, on the pathology report such that at least one state value in the first data set is obtained.

8. The method of claim 7, wherein the classification task is performed to obtain a state values of following fields: EGFR, ALK, ROS1, KRAS, BRAF, RET, NTRK, MET, P53, or Her2.

9. The method of claim 1, wherein the obtaining the first data set comprises:
performing a sequence tagging task, by a pre-trained model, on the pathology report such that at least one description in the first data set is obtained.

10. The method of claim 9, wherein the sequence tagging task is performed to obtain descriptions of following fields: operation, histology, tumor size, stage, PDL1.

11. The method of claim 9, wherein the pre-trained model is trained by a masked language model and/or a next sentence prediction.

12. A device of matching clinical trials, comprising:
a processor; and
a memory coupled with the processor,
wherein the processor executes computer-readable instructions stored in the memory to perform operations, and the operations comprise:
obtaining a first data set from a pathology report;
obtaining a second data set of a clinical trial;
determining a relevance value between the first data set and the second data set with respect to a first set of fields; and
determining the clinical trial is recommended when the relevance value exceeds a threshold.

13. The device of claim 12, further comprising:
determining whether a first data set and a second data set are matched with respect to a second set of fields,
wherein the relevance value is determined when the first data set and the second data set are matched with respect to the second set of fields.

14. The device of claim 12, wherein the relevance value is a sum of an individual relevance value of each of the first set of fields.

15. The device of claim 14, wherein the individual relevance value is associated with a respective inverse document frequency (IDF) for a respective keyword.

16. The device of claim 12, wherein the second set of fields include one or more of: estimated glomerular filtration rate (EFGR), surgical operation, histology, pathologic staging, age, gender, or smoking.

17. The device of claim 12, wherein the first set of fields include one or more of: ALK, ROS1, KRAS, BRAF, RET, NTRK, MET, P53, Her2, tumor size, tumor maximum diameter, programmed death-ligand 1 (PD-L1), nodal metastases, Distant metastases, CNS metastases, bone metastases, wild type, Anti-angiogenesis, Platinum, EGFR TKIs, ALK inhibitors, PD-1/PD-L1 inhibitors, CTLA-4 inhibitor, Radiotherapy, cisplatin/carboplatin, Chemotherapy, systemic therapy, Disease status, or Eastern Cooperative Oncology Group Performance Status (ECOG PS).

18. The device of claim 12, wherein the obtaining the first data set comprises:
performing a classification task, by a pre-trained model, on the pathology report such that at least one state value in the first data set is obtained.

19. The device of claim 12, wherein the obtaining the first data set comprises:
performing a sequence tagging task, by a pre-trained model, on the pathology report such that at least one description in the first data set is obtained.

20. A non-transitory computer storage medium having stored thereon program instructions that, upon execution by a processor, cause the processor to perform operations, comprising:
obtaining a first data set from a pathology report;
obtaining a second data set of a clinical trial;
determining whether the first data set and the second data set are matched with respect to a first set of fields;
determining a relevance value between the first data set and the second data set with respect to a second set of fields when the first data set and the second data set are matched with respect to the first set of fields; and
determining the clinical trial is recommended when the relevance value exceeds a threshold.
